(19) United States
(12) Patent Application Publication
(10) Pub. No.: US 2009/0022675 A1
(43) Pub. Date: Jan. 22, 2009

(54) ESTERS WITH WARMING PROPERTIES

(76) Inventor: EDWARD T. BORISH, Westerville, OH (US)

Correspondence Address: LAW OFFICES OF MITCHELL P. NOVICK, 66 PARK STREET, MONTCLAIR, NJ, 07042, US

(21) Appl. No.: 12/177,139

(22) Filed: Jul. 21, 2008

Related U.S. Application Data
(60) Provisional application No. 60/951,076, filed on Jul. 20, 2007.

(51) Int. Cl. A61K 8/37; A61Q 19/10; A61K 31/22; A61Q 17/04; A61Q 9/02

(52) U.S. Cl. 424/59; 510/130; 510/119; 514/506

(57) ABSTRACT

A heat-generating composition comprises a substituted polyhydric alcohol and a fatty acid ester, the composition having the structure of:

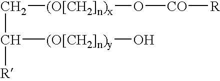

wherein R′ is H, CH₃—(O[CH₂]ₙ)_{z}—OH, or

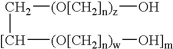

and wherein x+y, x+y+z or w+x+y+z, or combinations thereof, has an average value ranging from about 6 to about 100; wherein R is a hydrocarbon or mixture of alkyl groups comprising from about 3 to about 19 carbons, wherein n is an integer ranging from about 2 to about 4, or combinations thereof; and m is an integer ranging from about 0 to about 6. Interaction of the composition with an aqueous composition generates heat, characterized by a temperature increase from about 5 degrees C. to about 15 degrees C. Products containing the heat-generating composition can be applied to a surface in a two-step process, the first step being application of the composition, followed by the aqueous composition. The heat-generating composition can be used in cosmetics and personal care products.

### CROSS REFERENCES TO RELATED APPLICATIONS

This application claims the benefit of U.S. Provisional Patent Application Ser. No. 60/951,076, filed 20 Jul. 2007, by the present inventor, the contents of which are incorporated by reference herein in their entirety.

### FIELD OF THE INVENTION

Embodiments of this invention include compositions and methods using alkoxylated esters of carboxylic acids and polyhydric alcohols for their warming properties and describe an improved composition and method for this purpose.

### BACKGROUND OF THE INVENTION

This invention concerns compositions and methods using certain alkoxylated esters of carboxylic acids and polyhydric alcohols for their warming properties.

Surprisingly, we find that alkoxylated esters of certain acids with polyhydric alcohols and optionally and in combination with alkoxylated polyhydric alcohols have significant warming properties that are superior to other commonly used materials such as glycerin or propylene glycol. For example, we find such warming properties in the glycerol ester of the 8 carbon acid, ethylhexanoic acid ethoxylated with 18 moles of ethylene oxide per mole of ester (glycereth-18ethylhexanoate, “G-18-O”).

Glycereth-18ethylhexanoate has the further advantages of being very water soluble having a value of HLB greater than 10, is totally non-irritating even when neat G-18-O is held in intimate contact with skin under occlusive conditions for 48 hours, and serves as an effective antimicrobial agent.

Sometimes when a health or beauty product is applied to the skin or hair of an individual, the application of that product may produce a cooling effect on that individual, and which cooling effect the individual may consider as being uncomfortable. Many individuals seek out treatments in spas, where they are treated with products such as hot rocks, hot mud, warm oils and lotions for the benefit that such warm products may provide. Other warming products, such as heat wraps or localized heat treatments, are used to relax stiff or sore muscles. Thus, it can be desirable to have a warming effect when a product is applied, for a beneficial effect such as the opening of pores for skin cleansing, facial masks or the like, which does not require the need to obtain specialized treatment, and which can be done by the individual user.

In the present invention, methods for the use of a heat-generating composition, in conjunction with aqueous compositions, are described.

### BRIEF SUMMARY OF THE INVENTION

In one embodiment, an ester with warming properties comprises a substituted polyhydric alcohol and an ester of a fatty acid, the composition having the structure:

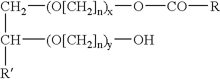

wherein R′ is H, CH₃—(O[CH₂]ₙ)_{z}—OH, or

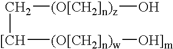

and wherein x+y, x+y+z or w+x+y+z, or combinations thereof, have an average value ranging from about 6 to about 100; wherein R is a hydrocarbon comprising from about 3 to about 19 carbons; wherein n is an integer ranging from about 2 to about 4 or combinations thereof; and wherein m is an integer ranging from 0 to about 6. The composition is water soluble, can be used in formulations, such as but not limited to cosmetics, household and personal care products, pharmaceuticals and pet care products. Personal care products include but are not limited to sanitizers, lotions, shampoos, anhydrous gels, sunscreens, hair products, moisturizing products, body massages, personal lubricants, shaving products and cleansers.

Other embodiments of this invention utilize alkoxylated esters of carboxylic acids and polyhydric alcohols for their warming properties in an improved composition and method for this purpose. The extent of alkoxylation is sufficient to increase the water solubility of the resultant material. In one embodiment of this invention, the polyhydric alcohol is glycerin and the carboxylic acid is ethylhexanoic acid. In another embodiment of this invention the composition is glycereth-18ethylhexanoate.

The beneficial warming properties of the instant invention are achieved by combining the heat generating G-18-O composition with water at the time of use. One method involves combining the heat generating G-18-O composition with an aqueous composition followed by application of the resultant mixture to a surface such as skin or hair of an individual. Another method involves the application of the heat-generating composition to a surface, such as the skin or hair of an individual. To the applied heat-generating composition is added an aqueous composition, such that the interaction between the two compositions generates heat at the surface. Another method involves the application of the heat-generating composition to a surface, such as the skin or hair of an individual to which an aqueous composition has been applied or which is otherwise moist, such that the interaction between the two compositions or the heat generating composition and moist surface generates heat at the surface. The two compositions are applied in any quantity where the concentration range by weight of each component is between 0% to 100%, and where the sum of the two compositions is 100%. The amount of heat generated is characterized by a temperature increase ranging from about 5 degrees C. to about 15 degrees C.

### BRIEF DESCRIPTION OF THE SEVERAL VIEWS OF THE DRAWING

FIG. 1 is a graph depicting the heat of solution of Hest G-18-O vs. Glycerine.

### DETAILED DESCRIPTION OF THE INVENTION

This invention involves using certain alkoxylated esters of carboxylic acids and polyhydric alcohols, the extent of ethoxylation being sufficient to make the resultant material substantially water soluble. Generally these are formed by the reaction of a carboxylic acid with an excess of a polyethylene glycol ether of a polyhydric alcohol. The reaction products are complex and may be formed by other methods known to those skilled in the art. For example only, and not intended to be a limitation, ethoxylation of glyceryl carboxylic acid esters may serve the same useful function. The resultant mixture here is primarily the alkoxylated esters of carboxylic acids and polyhydric alcohols with some residual polyethylene glycol ether of a polyhydric alcohol. Alkoxylation could be done using one or more compounds chosen from the group of oxiranes such as, but not limited to, ethylene oxide, propylene oxide, butylene oxide and the like. Ethylene oxide is employed in embodiments of the present invention. The polyhydric alcohol can be chosen from among but not limited to the 2 carbon to 10 carbon polyhydric alcohols. Among them are glycerol, polyethylene glycol, propylene glycol, ribitol and sorbitol. In an embodiment of the present invention, the polyhydric alcohol is glycerol. The ester conforms generally to the formula designated as Formula I:

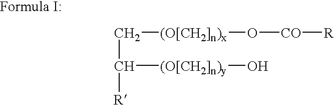

wherein R′ is H, CH₃—(O[CH₂]ₙ)_{z}—OH, or

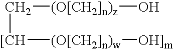

and wherein x+y, x+y+z or w+x+y+z, or combinations thereof, has an average value ranging from about 6 to about 100; wherein R is a hydrocarbon comprising from about 3 to about 19 carbons; wherein n is an integer ranging from about 2 to about 4 or combinations thereof; and wherein m is an integer ranging from 0 to about 6.

A product line designated as Hest represents various esters including alkoxylated esters of carboxylic acids and polyhydric alcohols. An exemplary material is sold under the trade name Hest G-18-O and is referred to by the INCI Name Glycereth-18Ethylhexanoate (and) Glycereth-18. Glycereth-18Ethylhexanoate has the molecular formula: C47H94022 with a molecular weight of 1010 grams per mole. Glycereth-18Ethylhexanoate is the ester of ethylhexanoic acid with a polyethylene glycol ether of glycerin containing an average of 18 moles of ethylene oxide and conforms generally to the formula shown below (Formula 2):

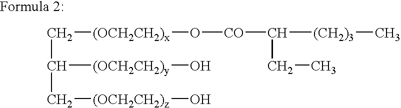

where x+y+z has an average value of 18.

The generic designation for glyceryl polyoxyethylene ethers is CAS No. 31694-55-0. Glycereth-18ethylhexanoate is assigned CAS No. 827307-65-6, and this composition is known to include glycereth-18 ethylhexanoate and glycereth-18; for purposes of this specification, the composition will be referred to as glycereth-18ethylhexanoate or G-18-O. Where the INCI name is used in the specification, the full INCI name will be stated as glycereth-18ethylhexanoate (and) glycereth-18. Both the term G-18-O and the INCI name are intended to refer to the same composition.

### Example 1

### Warming properties of glycereth-18-hexanoate.

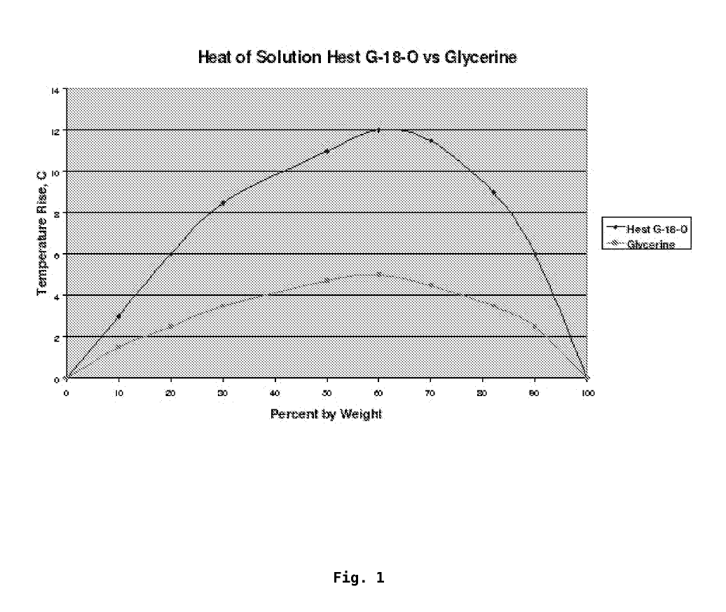

When Hest G-18-O is mixed with water at ambient temperature, there is a rise in temperature. The rise in temperature ranges from about 5 degrees C. to about 15 degrees C.; it also ranges from about 8 degrees C. to about 12 degrees C. This rise in temperature is about 2-3 times more than that obtained with the same weight of glycerine (FIG. 1) or propylene glycol. The temperature rise reaches a maximum of about 12 degrees C. when about 60% by weight of Hest G-18-O is used. This compares to a maximum of only about 5 degrees C. for glycerine or propylene glycol.

The maximum amount of heat (though not the greatest rise in temperature) is obtained when the Hest G-18-O is dissolved in a large excess of water. If the Hest G-18-O, glycerine or propylene glycol is not anhydrous, some of its heat of solution will already have been dissipated. Correspondingly, smaller amounts of heat will be produced by further dilution.

### Example 2

### Dermal safety testing of glycereth-18ethylhexanoate.

The dermal safety of G-18-O was tested using the neat (undiluted) material applied to the forearm of 51 human volunteers under occlusive conditions for 48 hours. These conditions are particularly aggressive, especially when compared with those in the prior art, where data on safety, if disclosed at all, is generally anecdotal using low concentrations of the agent. The dermal responses for G-18-O were scored by expert evaluators under clinical conditions according to the following 6-point scale:

0=No evidence of any effect;

+=Barely perceptible (Minimal, faint, uniform or spotty erythema);

1=Mild (Pink, uniform erythema covering most of the contact site);

2=Moderate (Pink-red erythema uniform in the entire contact site);

3=Marked (Bright re erythema with/without petechiae or papules); and

4=Severe (Deep red erythema with/without vesiculation or weeping).

Under these conditions G-18-O was totally non-irritating, with all panelists being scored as 0=No evidence of any effect. This indicates that G-18-O is exceptionally safe.

### Example 3

### Ocular safety testing of Glycereth-18Ethylhexanoate.

The ocular safety testing of G-18-O was tested using a 20% solution in water using the HET-CAM test. The test is a modification of that described by Kemper and Luepke in Kemper, F. H. & Luepke, H. P., (1986). The HET-CAM test: An Alternative to the Draize Test. FD Chem. Toxic. 24, p. 495-496. The chick embryo has been used extensively in toxicology. “The chorioallantoic membrane (CAM) of the chick embryo is a complete tissue with organois elements from all germ cell layers. The chorionic epithelium is ectodermal and the allantoic epithelium is endodermal. The mesoderm located between these epithelia is a complete connective tissue including arteries, capillaries, veins and lymphatic vessels. The CAM responds to injury with a complete inflammatory reaction, comparable to that in the rabbit eye test. It is technically easy to study, and without nerves to sense pain.” (Leighton, J., Tchao, R., Verdone, J. & Nassauer, J. Macroscopic Assay of Focal Injury in the Chorioallantoic Membrane. In: Alternative Methods in Toxicology, Vol. 3, In Vitro Toxicology E2, pp. 357-369, Alan M, Goldberg, (ed.), Mary Ann Liebert Publishers, Inc., N.Y., 1985.)

The ocular responses for G-18-O were classified according to the following scale.

| Score | Irritation Potential |
|---|---|
| 0.0-4.9 | Practically none |
| 5.0-9.9 | Slight |
| 10.0-14.9 | Moderate |
| 15.0-32.0 | Severe |

Under these conditions, the results indicate that G-18-O would have practically no ocular irritation potential in vivo being scored as 1.25. Under the same conditions, Johnson's Baby Shampoo was categorized as being moderately irritating being scored as 11.5. This indicates that G-18-O is exceptionally safe.

### Advantages

In various embodiments, the glycereth-18ethylhexanoate can be used in concentrations ranging from about 0.001% to about 100% by weight of the composition. In other embodiments, the glycereth-18ethylhexanoate can be used in concentrations ranging from about 10% to about 90% by weight of the composition, and in other embodiments, the glycereth-18ethylhexanoate can be used in concentrations ranging from about 30% to about 80% by weight of the composition, and in still other embodiments, the glycereth-18 ethylhexanoate can be used in concentrations ranging from about 50% to about 70% by weight of the composition.

As will be described further below, embodiments of G-18-O can be used in a number of different products and compositions. The composition of G-18-O can also be used as an antimicrobial, either alone or by being added to another solution, such as water, as described in a copending U.S. Pat. application, Ser. No. 11/714,986. The composition can also be used in a variety of products, such as but not limited to cosmetics, household products, personal care products for both humans and animals.

The warming effects of embodiments of the present invention can be utilized in a variety of health and beauty products. Such products can include, for example only, lotions, creams or oils intended for use in massaging of the body, hair and scalp treatments, shampoos, hair conditioners, shaving cream and related products, facial masks, body massages, personal lubricants, or other product forms such as creams, dispersions, gels, ointments, sprays, sticks, powders, suspensions and the like.

A multi-step application process can be used to achieve the greatest heat-generating effect. In one embodiment, the first step is the application of an anhydrous preparation of G-18-O to the selected area. This is followed by the application of one or more aqueous compositions, which upon interaction with the applied G-18-O, will generate the warming effect. Another method involves the application of the heat-generating composition to a surface, such as the skin or hair of an individual to which an aqueous composition has been applied or which is otherwise moist, such that the interaction between the two compositions or the heat generating composition and the moist surface generates heat at the surface. Another method involves combining the heat generating G-18-O composition with an aqueous composition followed by application of the resultant mixture to a surface such as skin or hair of an individual. The compositions could be applied to the selected site on either a weight:weight basis, or on a volume:volume basis. For most applications, the compositions would be applied using a volume:volume basis. Thus, for example, a quantity of the G-18-O preparation is first applied to the selected area of the body, and then a quantity of a second composition, such as, for example, a lotion, is then applied over the G-18-O preparation, such that the two compositions become mixed, and the interaction of the compositions produces the desired heating effect on the individual.

While the aqueous formulations shown in the Examples recite the final concentration of the G-18-O in a completed product, it is anticipated that the use of the inventive methods will require manufacturing of these formulations in two containers, one containing the G-18-O, the second containing the remainder of the composition, such that when mixed together at the selected area, the mixing will generate heat and complete the formulation. Depending upon manufacturing concerns, a third container, for use in mixing the two compositions prior to application, could be provided.

In some formulations, it may be necessary to increase the viscosity of the composition to enable it to adhere to a surface, or to improve how the composition feels to one's touch when it is being used or applied. One embodiment utilizes a thickening agent of sufficient yield to stabilize the dispersion until use. Other ingredients such as, but not limited to, preservatives, colorants and fragrance can be added to the composition as desired.

Depending upon its ultimate use, the pH of the G-18-O may be adjusted initially, or, as it is more commonly done, the pH of the formulation is adjusted during the manufacturing process. For pH adjustment, any agent commonly used in the laboratory for pH adjustment, such as hydrochloric acid or sodium hydroxide, can be used. Other acids such as acetic acid, benzoic acid, formic acid, fumaric acid, lactic acid, phosphoric acid, sulfuric acid or other organic and/or inorganic acids, as known to those skilled in the art, could also be used. Other bases such as ammonium hydroxide, ethanolamine, magnesium hydroxide, sodium or potassium bicarbonate, sodium or potassium hydroxide, organic and/or inorganic bases, as known to those skilled in the art, could also be used. Depending upon the particular viscosifier employed in the compositions, other agents for adjusting pH could also be used. A propanol derivative, such as aminomethyl propanol, may be used to adjust the pH when a CARBOPOL7 (registered trademark of Noveon, Inc., Cleveland, Ohio) is used as the viscosity modifying agent.

To facilitate maintaining an active agent in suspension during storage, shipment or prior to use, one or more of a number of viscosity modifying agents (“viscosifiers”) can be added to the composition. For example only, and not intended as any limitation, examples of viscosifiers that could be utilized include carageenans; cellulose compounds such as methyl cellulose, hydroxymethyl cellulose, and carboxymethyl cellulose; pectin; dextrans of various molecular weight ranges; starch; gum tragacanth; gum arabic; guar gum; acacia gum; gum karaya; silica, diatomaceous earth; and other commonly used agents known to those skilled in the art. The viscosifier is generally added to the composition in the range recommended by its manufacturer, and in an embodiment of the present invention, this generally ranges from about 0.1% to about 1.0%. One example of a viscosifier that can be used is an Acrylate/C10-30 Alkyl Acrylate Crosspolymer (CTFA-INCI nomenclature); it is a member of the class of viscosifiers sold commercially under the trade name CARBOPOL7 (registered trademark of Noveon, Inc., Cleveland, Ohio). In other embodiments, the range can be from about 0.001% to about 10%, depending upon the combination of viscosifier employed, and the other agents contained within the composition, as recommended by the product manufacturers, and known to those skilled in the art.

One or more preservatives can be added to the composition. Examples of such preservatives are butylated hydroxyanisole (“BHA”), butylated hydroxytoluene (“BHT”), phenol, resorcinol; parabens such as methyl paraben, ethyl paraben, propyl paraben, butyl paraben, isopropyl paraben and isobutyl paraben and the like; 2-phenoxyethanol, 1,3-Octandiol; sodium benzoate, benzyl alcohol, or other preservatives commonly used in the industry. Other phenolic agents include 4,6-di-tert-butyl-resorcinol, 2,6-di-tert-butylphenol, 2,5-di-tert-butylphenol, 3,5-di-tert-butylphenol, 2,6-di-tert-hexylphenol, 2,6-di-tert-octylphenol and 2,6-di-tert-decylphenol. The general range for addition of preservatives is from about 0.01% to about 10% by weight of the composition. In one embodiment of the present invention, the preservative is added in the range of about 0.01% to about 1% by weight of the composition, and in another embodiment the preservative is added in the range of about 0.1% to about 0.6% by weight of the composition.

In one embodiment of the present invention, the preservative used is 1,3-Dimethylol-5,5-Dimethyl Hydantoin (CFTA-ICNI nomenclature of DMDM Hydantoin), and is sold commercially under the trade name GLYDANT7 (registered trademark of Lonza, Inc., Fair Lawn, N.J.), or under the trademark MACKSTAT7 DM (registered trademark of McIntyre Group Ltd., University Park, Ill.), and used in accordance with the manufacturer's specifications.

One or more agents to prevent or reduce irritation can be added to the composition. One example of an anti-irritant agent is polyvinylpyrrolodine.

A colorant can be added to the composition if desired. One or more colorants can be added to the composition, and without specifying any one in particular, it is to be understood that these colorants can be obtained from various commercial sources including but not limited to those list in the CTFA INCI Dictionary all of which are incorporated here by reference. The colorant can be added to the composition in a range from about 0.00001% to about 5%, as known to those skilled in the art. In one embodiment of the present invention, the colorant is used in the range of about 0.0001% to about 0.0005%.

A fragrance can be added to the composition if desired. One or more fragrances can be added to the composition, and without specifying any one in particular, it is to be understood that these fragrances can be obtained from various commercial sources, such as, for example only, International Flavors and Fragrances (New York, N.Y.). The fragrance can be added to the composition in a range from about 0.001% to about 20%, as known to those skilled in the art. Examples of fragrances that could be used include, for example only and are not intended to be limited, ammonium glycyrrhizate, amyl acetate, anisaldehyde, benzoic acid, betula alba extract, caraway fruit oil, safflower seed oil, caramel, cedarwood oil, cinnamyl acetate, citrus extracts such as from orange, grapefruit, lemon or lime, citronella, carrot, clove, eucalyptus, wintergreen, licorice, lavender, cherry, various berries or the like.

### Example 4

### Body Massage and Personal Lubricant

| | | Concentration |
|---|---|---|
| INGREDIENT | INCI NAME | by weight |
| Glycerin | Glycerin | 0.50% |
| Propylene glycol | Propylene glycol | 0.50% |
| Hest G-18-0 | Glycereth-18 Ethylhexanoate | 99.00% |
| | (and) Glycereth-18 | |
| TOTAL | | 100.00% |

The composition is prepared by combining the Hest G-18-O, Glycerin and Propylene glycol and mixing well.

### Example 5

### Sugar Scrub

| | | Concentration |
|---|---|---|
| INGREDIENT | INCI NAME | by weight |
| Drakeol7 10 | Mineral Oil | 15.50% |
| Light Global 4075 | Glyceryl Isostearate (and) | 13.80% |
| | Caprylic/Capric Glycerides | |
| Hest G-18-0 | Glycereth-18 Ethylhexanoate | 5.00% |
| | (and) Glycereth-18 | |
| Sugar | Sucrose | 63.00% |
| Sodium Stearate | Sodium Stearate | 2.70% |
| TOTAL | | 100.00% |

The composition is prepared by mixing the Global 4075, glycereth-18ethylhexanoate and sodium stearate, and heating the mixture until it is homogeneous. The mineral oil (Drakeol7 10, registered trademark of Penreco, Division of Pennzoil Inc., Butler, Pa.) is heated to the same temperature in a separate vessel. The mineral oil is then added to the mixture of Global 4075, Hest G-18-O, and sodium stearate, followed by the sugar and cooling the resultant mixture to room temperature.

### Example 6

### Anhydrous Gel

| | | Concentration |
|---|---|---|
| INGREDIENT | INCI NAME | by weight |
| Drakeol7 10 | Mineral Oil | 70.00% |
| Light Global 4075 | Glyceryl Isostearate (and) | 20.00% |
| | Caprylic/Capric Glycerides | |
| Hest G-18-0 | Glycereth-18 Ethylhexanoate | 5.00% |
| | (and) Glycereth-18 | |
| Sodium Stearate | Sodium Stearate | 5.00% |
| TOTAL | | 100.00% |

The composition is prepared by mixing the Global 4075, glycereth-18ethylhexanoate and sodium stearate, and heating the mixture until it is homogeneous. The mineral oil (Drakeol7 10, registered trademark of Penreco, Division of Pennzoil Inc., Butler, Pa.) is heated to the same temperature in a separate vessel. The mineral oil is then added to the mixture of Global 4075, Hest G-18-O, and sodium stearate, followed by cooling the resultant mixture to room temperature.

### Example 7

### Sunscreen Gel Stick

| | | Concentration |
|---|---|---|
| INGREDIENT | INCI NAME | by weight |
| Hest 25B | C12-C15 Alkyl Benzoate | 65.50% |
| Escalol7 557 | Octinoxate | 7.50% |
| Escalol7 587 | Octisal | 5.00% |
| Corapan7 TQ | Diethylhexyl 2,6 | 4.10% |
| | Naphthalate | |
| Global 4075 | Glyceryl Isostearate (and) | 20.00% |
| | Caprylic/Capric Glycerides | |
| Hest G-18-0 | Glycereth-18 Ethylhexanoate | 5.00% |
| | (and) Glycereth-18 | |
| Sodium Stearate | Sodium Stearate | 5.00% |
| TOTAL | | 100.00% |

The composition is prepared by mixing the Global 4075, Hest G-18-O, and sodium stearate, and heating the mixture until it is homogeneous. The other four ingredients are heated to the same temperature in a separate vessel. The contents of the second vessel is then added to the mixture of Global 4075, glycereth-18ethylhexanoate and sodium stearate, followed by cooling the resultant mixture to room temperature.

Corapan7 is a registered trademark of International Specialty Products, Wayne, N.J. for chemical preparations for use in the manufacture of personal care products.

Escalol7 is a registered trademark of International Specialty Products, Wayne, N.J. for ultraviolet absorbing compounds for use in the manufacture of skin care and hair care products.

### Example 8

### Moisturizing Hand Sanitizer

| | | Concentration |
|---|---|---|
| INGREDIENT | INCI NAME | by volume |
| Water | Water | 30.74% |
| Ultrez ® 21 | Acrylates/C10-30 Alkyl | 0.40% |
| | Acrylate Crosspolymer | |
| SD Alcohol 40-B | Alcohol Denatured (Ethyl | 63.00% |
| | Alcohol) | |
| AMP-95 | Aminoethyl Propanol | 0.36% |
| Hest G-18-0 | Glycereth-18 Ethylhexanoate | 5.50% |
| TOTAL | | 100.00% |

The sanitizer composition is prepared by slowly hydrating the Ultrez® 21 (registered trademark of Carbopol Corp.), followed by addition of the ethanol with mixing until the mixture is uniform. The aminoethyl propanol is then added and mixed until the mixture is uniform.

### Example 9

### Antibacterial Hand Lotion

| | | Concentration |
|---|---|---|
| INGREDIENT | INCI NAME | by volume |
| Water | Water | 22.65% |
| Ultrez ® 21 | Acrylates/C10-30 Alkyl | 0.80% |
| | Acrylate Crosspolymer | |
| SD Alcohol 40-B | Alcohol Denatured (Ethyl | 63.00% |
| | Alcohol) | |
| AMP-95 | Aminoethyl Propanol | 0.55% |
| Hest IS-3-O | Isosteareth-3 Ethylhexanoate | 10.00% |
| Hest G-18-0 | Glycereth-18 Ethylhexanoate | 3.00% |
| TOTAL | | 100.00% |

The hand lotion composition is prepared by slowly hydrating the Ultrez® 21, followed by addition of the ethanol with mixing until the mixture is uniform. The aminoethyl propanol is then added and mixed until the mixture is uniform, followed by the addition of the isosteareth-3 ethylhexanoate which is then mixed until the mixture is uniform.

### Example 10

### Amide-Free Shampoo Formulation

| | | Concentration |
|---|---|---|
| INGREDIENT | INCI NAME | by volume |
| Water | Water | 24.30% |
| Rhodapex ® EA-2 | Ammonium Laureth Sulfate | 50.00% |
| Rhodapon ® L-22 | Ammonium Lauryl Sulfate | 10.00% |
| Mirataine ® BET- | Cocoamidopropyl Betain | 5.00% |
| C 30 | | |
| Hetoxamate 6000 | PEG-150 Distearate | 0.50% |
| DS Special | | |
| Hest G-18-0 | Glycereth-18 Ethylhexanoate | 10.00% |
| Mackstat ® DMDM | DMDM Hydantoin | 0.20% |
| TOTAL | | 100.00% |

The shampoo composition is prepared by mixing the first three ingredients, and heating the mixture to about 70 degrees C. The Hetoxamate 6000 DS Special is added, and heating is continued to about 80 degrees C. The Mirataine® BET-C 30 is then added, and the mixture maintained at about 80 degrees C. for a period ranging from about 5 to about 15 minutes. The mixture is then allowed to cool towards room temperature, followed by the addition of the preservative (Mackstat) at a temperature of about 40-45 degrees C., after which the mixture is allowed to cool to room temperature.

### Example 11

### Hair Gel

| | | Concentration |
|---|---|---|
| INGREDIENTS | INCI NAME | by volume |
| Water | Water | 89.55% |
| Ultrez 21 | Acrylates/C10-30 Alkyl Acrylate | 1.00% |
| | Crosspolymer | |
| Fixative G-100 | AMP-Acrylates/Allyl Methacrylate | 3.50% |
| Polymer | Copolymer | |
| Hest G-18-0 | Glycereth-18 Ethylhexanoate (and) | 5.00% |
| | Glycereth-18 | |
| AMP-95 | Aminomethyl Propanol | 0.95% |
| TOTAL | | 100.00% |

### Example 12

### Moisturizing Cream

| | | Concentration |
|---|---|---|
| INGREDIENTS | INCI NAME | by volume |
| Hest P-4O | Pentaerythrityl Tetraoctanoate | 7.00% |
| Hest ICS | Isocetyl Stearate | 7.00% |
| Hest 25B | C12-15 Alkyl Benzoate | 7.00% |
| Hetoxol STA-2 | Steareth-2 | 5.00% |
| Hetoxol I-20-20 | Octyldodeceth-20 | 1.00% |
| Hest G-18-O | Glycereth-18 Ethylhexanoate | 1.00% |
| | (and) Glycereth-18 | |
| Water | Water | 72.00% |
| TOTAL | | 100.00% |

The moisturizing cream emulsion composition is prepared by combining the Hest 25B, Hest ICS, Hest P-40, Hetoxol STA-2 and Hetoxol I-20-20 and heating the mixture to 80-85 degrees C. In a separate vessel, the water is heated to the same temperature. With adequate mixing, step 1 is slowly added to step 2 and mixing is continued for 10 minutes after which the mixing speed is reduced the composition is cooled to room temperature.

### Example 13

### Moisturizing Mist & Mousse

| | | Concentration |
|---|---|---|
| INDGREDIENTS | INCI NAME | by volume |
| Water | Water | 89.50% |
| Hest G-18-O | Glycereth-18 Ethylhexanoate | 10.00% |
| | (and) Glycereth-18 | |
| Glycerin | Glycerin | 0.50% |
| TOTAL | | 100.00% |

This moisturizing composition is prepared by combining the Water and Glycerin and mixing till uniform. The composition is delivered using a pump spray or an Airfoamer dispenser.

### Example 14

### Moisturizing Lotion

| | | Concentration |
|---|---|---|
| INGREDIENTS | INCI NAME | by volume |
| Hest 25B | C12-15 Alkyl Benzoate | 5.00% |
| Hest TC | Tricaprylin | 10.00% |
| Hest T-3S | Trimethylolpropane tristearate | 3.00% |
| Hest P-4IS | Pentaerythrityl isostearate | 6.00% |
| HetoxolI-20-20 | Octyldodeceth-20 | 2.00% |
| Hetoxol STA-2 | Steareth-2 | 6.00% |
| Hest G-18-O | Glycereth-18 Ethylhexanoate (and) | 5.00% |
| | Glycereth-18 | |
| Carbopol | Acrylates/C10-30 alkyl | 0.20% |
| Triethanolamine | Triethanolamine | 0.30% |
| Water | Water | 62.50% |
| TOTAL | | 100.00% |

The moisturizing lotion emulsion composition is prepared by combining the Hest 25B, Hest TC, Hest P-4IS, Hest T-3S, Hetoxol STA-2, and Hetoxol I-20-20 and heating to 80-85 degrees C. In a separate vessel, Carbopol Ultrez-21 is slowly hydrated and the mixture is heated to the same temperature. With adequate mixing step 1 is slowly added to step 2 and mixing is continued for 10 minutes after which the mixing speed is reduced and the composition is cooled to room temperature.

### Example 15

### Self Foaming Cleanser

| | | Concentration |
|---|---|---|
| INGREDIENTS | INCI NAME | by volume |
| Water | Water | 82.00% |
| Mackam 2C | Disodium Cocoamphodiacetate | 12.00% |
| Hest G-18-O | Glycereth-18 Ethylhexanoate (and) | 2.00% |
| | Glycereth-18 | |
| Hetoxide G-26 | Glycereth-26 | 2.00% |
| Hetoxide G-7 | Glycereth-7 | 2.00% |
| TOTAL | | 100.00% |

The cleanser composition is prepared by combining the Water, Mackam 2C, Glycereth-26 and Glycereth-7 and mixing till uniform. The composition is delivered using an Airfoamer dispenser.

The chemical formula of one embodiment of the composition is shown below, as Formula (3):

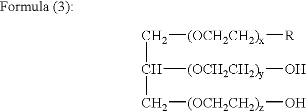

where R is an alkyl group or mixture of alkyl groups having a chain length less than C10; and

x, y, and z is the average number of ethylene oxide units in the polyhydric alcohols segment of the molecule, and where the sun of x plus y plus z has an average value of 18. Each of x, y, and z is a numerical value between 1 & 8 inclusive.

While certain examples, such as those for the moisturizing hand sanitizer and the moisturizing cream or lotion, omit specific addition of G-18-O in their method of preparation, it is to be understood that when the single step application method is practiced, the formulation added to the moist surface is a complete formulation containing glycereth-18-ethylhexanoate at the appropriate concentration.

Therefore, although this invention has been described with a certain degree of particularity, it is to be understood that the present disclosure has been made only by way of illustration, and that numerous changes in the details of the composition and additives may be resorted to without departing from the spirit and scope of the invention.

I claim:

1. A method of generating heat at a surface, the method comprising the steps of:
applying a quantity of a heat-generating composition to the surface, the heat-generating composition comprising:
a substituted polyhydric alcohol and an ester of a carboxylic acid, the heat-generating composition having the structure of: 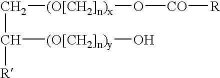
wherein R′ is H, CH₃—(O[CH₂]ₙ)_{z}—OH, or 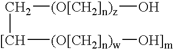
and wherein x+y, x+y+z or w+x+y+z, or combinations thereof, has an average value ranging from about 6 to about 100; wherein R is a hydrocarbon or mixture of alkyl groups comprising from about 3 to about 19 carbons, wherein n is an integer ranging from about 2 to about 4, or combinations thereof; and m is an integer ranging from about 0 to about 6; and
applying a quantity of an aqueous composition to the applied heat-generating composition, the interaction between the heat-generating composition and the aqueous composition generating heat at the surface.

2. The method as described in claim 1, wherein the heat-generating composition comprises from about 0.001% to about 100% by weight of the applied compositions.

3. The method as described in claim 2, wherein the heat-generating composition comprises from about 0.001% to about 10% by weight of the applied compositions.

4. The method as described in claim 3, wherein the heat-generating composition comprises from about 0.01% to about 8% by weight of the applied compositions.

5. The method as described in claim 2, wherein the heat generated is characterized by a temperature increase ranging from about 5 degrees C. to about 15 degrees C.

6. The method as described in claim 2, wherein the heat generated is characterized by a temperature increase ranging from about 8 degrees C. to about 12 degrees C.

7. The method as described in claim 1, wherein in the heat-generating composition, each of w, x, y, and z has a numerical value between about 1 and about 50.

8. The method as described in claim 7, wherein in the heat-generating composition, R comprises alkyl groups having carbon chain lengths ranging from C4 to C10.

9. The method as described in claim 8, wherein R comprises alkyl groups ranging from C6 to C8.

10. The method as described in claim 2, wherein the heat-generating composition has the formula:

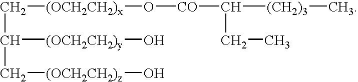

11. The method as described in claim 10, wherein in the heat-generating composition, the polyhydric alcohol is selected from the group consisting of glycerol, polyethylene glycol, ribitol and sorbitol.

12. The method as described in claim 2, wherein the aqueous composition is selected from the group consisting of cosmetics and personal care products.

13. The method as described in claim 12, wherein the personal care product is selected from the group consisting of a sanitizer, a hand lotion, a shampoo, a gel, a sunscreen, a hair gel, a moisturizing product, a moisturizing cream, a moisturizing mist, a facial mask, a mousse, a moisturizing lotion, a shave cream, a body massage, a personal lubricant and a self foaming cleanser.

14. A method of generating heat at a surface, the method comprising the steps of:
applying a quantity of a heat-generating composition to the surface, wherein the heat-generating composition has the formula: 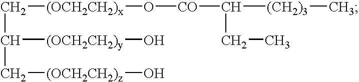
wherein x+y or x+y+z, or combinations thereof, has an average value ranging from about 6 to about 100; and
applying a quantity of an aqueous composition to the applied heat-generating composition, whereby the interaction between the heat-generating composition and the aqueous composition generates heat at the surface.

15. The method as described in claim 14, wherein the compositions are applied to the skin or hair of a mammal.

16. A method of generating heat at a surface, the method comprising the steps of:
applying a quantity of a heat-generating composition to a moist surface, the heat-generating composition comprising:
a substituted polyhydric alcohol and an ester of a carboxylic acid, the heat-generating composition having the structure of: 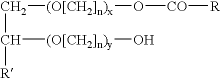
wherein R′ is H, CH₃—(O[CH₂]ₙ)_{z}—OH, or 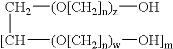
and wherein x+y, x+y+z or w+x+y+z, or combinations thereof, has an average value ranging from about 6 to about 100; wherein R is a hydrocarbon or mixture of alkyl groups comprising from about 3 to about 19 carbons, wherein n is an integer ranging from about 2 to about 4, or combinations thereof; and m is an integer ranging from about 0 to about 6, the interaction between the heat-generating composition and the moist surface generates heat at the surface.

17. The method as described in claim 16, wherein the heat-generating composition further comprises a composition selected from the group consisting of a skin sanitizer, a shampoo, a hand lotion, a sun screen, an anhydrous gel, a cosmetic and a personal care product.

18. A method for applying heat to a subject in need thereof, the method comprising the steps of:
applying to the subject an amount of a composition comprising a substituted polyhydric alcohol and an ester of a carboxylic acid, the composition having the structure of: 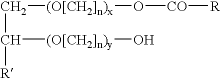
wherein R′ is H, CH₃—(O[CH₂]ₙ)_{z}—OH, or 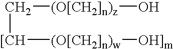
and wherein x+y, x+y+z or w+x+y+z, or combinations thereof, has an average value ranging from about 6 to about 100; wherein R is a hydrocarbon or mixture of alkyl groups comprising from about 3 to about 19 carbons, wherein n is an integer ranging from about 2 to about 4, or combinations thereof; and m is an integer ranging from about 0 to about 6; and
applying a quantity of an aqueous composition to the applied heat-generating composition, the interaction between the heat-generating composition and the aqueous composition generating heat and applying the heat to the subject.

19. The method as described in claim 18, further comprising the step of adding the composition to a formulation, the formulation being chosen from the group consisting of a skin sanitizer, a shampoo, a hand lotion, a sun screen, an anhydrous gel, a cosmetic and a personal care product.

20. A composition for generating heat, the composition comprising:
a substituted polyhydric alcohol and an ester of a carboxylic acid, the heat-generating composition having the structure of: 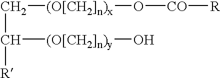
wherein R′ is H, CH₃—(O[CH₂]ₙ)_{z}—OH, or 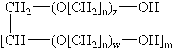
and wherein x+y, x+y+z or w+x+y+z, or combinations thereof, has an average value ranging from about 6 to about 100; wherein R is a hydrocarbon or mixture of alkyl groups comprising from about 3 to about 19 carbons, wherein n is an integer ranging from about 2 to about 4, or combinations thereof; and m is an integer ranging from about 0 to about 6, the interaction between the heat-generating composition and an aqueous agent generates heat.

21. The composition as described in claim 20, wherein the heat-generating composition further has the formula: 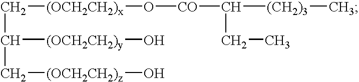
wherein x+y or x+y+z, or combinations thereof, has an average value ranging from about 6 to about 100.
